(19) United States
(12) Patent Application Publication
(10) Pub. No.: US 2010/0272318 A1
(43) Pub. Date: Oct. 28, 2010

(54) ENDOSCOPIC MEASUREMENT TECHNIQUES

(75) Inventors: Oz Cabiri, Macabim (IL); Tzvi Philipp, Beit Shemesh (IL); Boaz Shpigelman, Netanya (IL); Daniel Goldstein, Efrat (IL)

Correspondence Address: FROMMER LAWRENCE & HAUG, 745 FIFTH AVENUE- 10TH FL., NEW YORK, NY, 10151, US

(73) Assignee: G.I. VIEW LTD

(21) Appl. No.: 11/914,377

(22) PCT Filed: May 11, 2006
(86) PCT No.: PCT/IL06/00563

§ 371 Date: Nov. 19, 2008

Related U.S. Application Data
(60) Provisional application No. 60/680,599, filed on May 13, 2005.

(51) Int. Cl. G06K 9/00; A61B 1/04

(52) U.S. Cl. 382/106; 600/109

(57) ABSTRACT

Apparatus for use in a lumen is provided, including a light source, configured to illuminate a vicinity of an object of interest of a wall of the lumen, and an optical system (20), which is configured to generate a plurality of images of the vicinity. The apparatus further includes a control unit, which configured to measure a first brightness of a portion of a first one of the plurality of images generated while the optical system (20) is positioned at a first position with respect to the vicinity, measure a second brightness of a portion of a second one of the plurality of images generated while the optical system (20) is positioned at a second position with respect to the vicinity, the second position different from the first position, wherein the portion of the second one of the images generally corresponds to the portion of the first one of the images, and calculate a distance to the vicinity, responsively to the first and second brightnesses. Other embodiments are also described.

### CROSS-REFERENCES TO RELATED APPLICATIONS

The present application claims the benefit of U.S. Provisional Patent Application 60/680,599, filed May 13, 2005, entitled, “Endoscopic measurement techniques,” which is assigned to the assignee of the present application and is incorporated herein by reference.

### FIELD OF THE INVENTION

The present invention relates generally to medical devices, and specifically to endoscopic medical devices.

### BACKGROUND OF THE INVENTION

Medical endoscopes are used to inspect regions within the body, such as cavities, organs, and joints. Endoscopes typically include a rigid or flexible elongated insertion tube having a set of optical fibers that extend from a proximal handle through the insertion tube to the distal viewing tip of the endoscope. Alternatively, an image sensor, such as a CCD, is positioned near the distal viewing tip. An external or internal light source provides light to the area of interest in the body in the vicinity of the distal tip.

US Patent Application Publication 2004/0127785 to Davidson et al., which is incorporated herein by reference, describes techniques for capturing in-vivo images and enabling size or distance estimations for objects within the images. A scale is overlayed on or otherwise added to the images and, based on a comparison between the scale and an image of an object, the size of the object and/or the distance of the object from an imaging device is estimated or calculated. Also described are techniques for determining the approximate size of an object by knowing the size of a dome of the device and an illumination range of the illumination device. In an embodiment, a method for determining the distance of an object includes measuring the intensity of reflected illumination from the object, and correlating the illumination with the object's distance from the device. Such distance is used to calculate the estimated size of the object.

U.S. Pat. No. 5,967,968 to Nishioka, which is incorporated herein by reference, describes an endoscope comprising a distal end, an instrument channel extending therethrough, and a lens at the distal end adjacent the instrument channel; and an elongate probe configured to be inserted through the instrument channel and contact an object of interest. The probe comprises a plurality of unevenly spaced graduations along its length, each graduation indicating a size factor used to scale the image produced by the endoscope.

U.S. Pat. No. 4,721,098 to Watanabe, which is incorporated herein by reference, describes an inserting instrument that is insertable through an inserting portion of an endoscope so as to have a distal end portion projected from a distal end of the inserting portion. The inserting instrument comprises an outer tubular envelope and an elongated rod-like member located at a distal end of the envelope. An operating device located at a proximal end of the envelope is connected to the rod-like member through a wire member extending through the envelope. The rod-like member is operated to be moved between an inoperative position where the longitudinal axis of the rod-like member extends substantially in coaxial relation to the envelope and an operative position where the longitudinal axis of the rod-like member extends across an extended line of the envelope. The inserting instrument may be utilized as a measuring instrument, in which case the rod-like member has carried thereon graduations for measurement.

PCT Publication WO 03/053241 to Adler, which is incorporated herein by reference, describes techniques for calculating a size of an object using images acquired by a typically moving imager, for example in the GI tract. A distance traveled by the moving imager during image capture is determined, and spatial coordinates of image pixels are calculated using the distance. The size of the object is determined, for example, from the spatial coordinates. The moving imager may be contained in a swallowable capsule or an endoscope.

US Patent Application Publication 2004/0008891 to Wentland et al., which is incorporated herein by reference, describes techniques for analyzing known data, and storing the known data in a pattern database (“PDB”) as a template. Additional methods are described for comparing target data against the templates in the PDB. The data is stored in such a way as to facilitate the visual recognition of desired patterns or indicia indicating the presence of a desired or undesired feature within the new data. The techniques are described as being applicable to a variety of applications, including imaging of body tissues to detect the presence of cancerous tumors.

PCT Publication WO 02/075348 to Gal et al., which is incorporated herein by reference, describes a method for determining azimuth and elevation angles of a radiation source or other physical objects located anywhere within an cylindrical field of view. The method uses an omni-directional imaging system including reflective surfaces, an image sensor, and an optional optical filter for filtration of the desired wavelengths. Use of two such systems separated by a known distance, each providing a different reading of azimuth and elevation angle of the same object, enables classic triangulation for determination of the actual location of the object.

The following patents and patent application publications, all of which are incorporated herein by reference, may be of interest:

U.S. Pat. No. 5,710,661 to Cook

U.S. Pat. No. 6,341,044 to Driscoll, Jr. et al.

U.S. Pat. No. 6,493,032 and US Patent Application Publication 2002/0012059 to Wallerstein et al.

U.S. Pat. No. 6,356,296 to Driscoll, Jr. et al.

U.S. Pat. Nos. 6,459,451 and 6,424,377 to Driscoll, Jr. et al.

U.S. Pat. No. 6,373,642 to Wallerstein et al.

U.S. Pat. No. 6,388,820 to Wallerstein et al.

U.S. Pat. No. 6,597,520 to Wallerstein et al.

U.S. Pat. No. 4,647,761 to Cojan et al.

U.S. Pat. No. 5,790,182 to St. Hilaire

U.S. Pat. No. 6,130,783 to Yagi et al.

U.S. Pat. No. 6,646,818 to Doi

U.S. Pat. No. 6,222,683 to Hoogland et al.

U.S. Pat. No. 6,304,285 to Geng

U.S. Pat. No. 5,473,474 to Powell

U.S. Pat. No. 5,920,376 to Bruckstein et al.

U.S. Pat. No. 6,375,366 to Kato et al.

U.S. Pat. No. 5,739,852 to Richardson et al.

U.S. Pat. No. 6,115,193 to Shu

U.S. Pat. No. 5,502,592 to Jamieson

U.S. Pat. No. 4,012,126 to Rosendahl et al.

U.S. Pat. No. 6,028,719 to Beckstead et al.

U.S. Pat. No. 6,704,148 to Kumata

U.S. Pat. No. 4,976,524 to Chiba

U.S. Pat. No. 6,611,282 to Trubko et al.

U.S. Pat. No. 6,333,826 to Charles

U.S. Pat. No. 6,449,103 to Charles

U.S. Pat. No. 6,157,018 to Ishiguro et al.

US Patent Application Publication 2002/0109773 to Kuriyama et al.

US Patent Application Publication 2002/0109772 to Kuriyama et al.

US Patent Application 2004/0004836 to Dubuc

US Patent Application Publication 2004/0249247 to Iddan

US Patent Application Publication 2003/0191369 to Arai et al.

PCT Publication WO 01/68540 to Friend

PCT Publication WO 02/059676 to Gal et al.

PCT Publication WO 03/026272 to Gal et al.

PCT Publication WO 03/046830 to Gal et al.

PCT Publication WO 04/042428 to Gal et al.

PCT Publication WO 03/096078 to Gal

PCT Publication WO 03/054625 to Gal et al.

PCT Publication WO 04/008185 to Gal et al.

Japanese Patent Application Publication JP 61-267725 A2 to Miyazaki Atsushi

Japanese Patent Application Publication JP 71-91269 A2 to Yamamoto Katsuro et al.

### SUMMARY OF THE INVENTION

In embodiments of the present invention, an optical system for use with a device comprises an optical assembly and an image sensor, such as a CCD or CMOS sensor. Typically, the device comprises an endoscope for insertion in a lumen. For some applications, the endoscope comprises a colonoscope, and the lumen includes a colon of a patient. The optical system is typically configured to enable forward and omnidirectional lateral viewing.

In an embodiment, the optical system is configured for use as a gastrointestinal (GI) tract screening device, e.g., to facilitate identification of patients having a GI tract cancer or at risk for same. Although for some applications the endoscope may comprise an element that actively interacts with tissue of the GI tract (e.g., by cutting or ablating tissue), typical screening embodiments of the invention do not provide such active interaction with the tissue. Instead, the screening embodiments typically comprise passing an endoscope through the GI tract and recording data about the GI tract while the endoscope is being passed therethrough. (Typically, but not necessarily, the data are recorded while the endoscope is being withdrawn from the GI tract.) The data are analyzed, and a subsequent procedure is performed to actively interact with tissue if a physician or algorithm determines that this is appropriate.

It is noted that screening procedures using an endoscope are described by way of illustration and not limitation. The scope of the present invention includes performing the screening procedures using an ingestible capsule, as is known in the art. It is also noted that although omnidirectional imaging during a screening procedure is described herein, the scope of the present invention includes the use of non-omnidirectional imaging during a screening procedure.

For some applications, a screening procedure is provided in which optical data of the GI tract are recorded, and an algorithm analyzes the optical data and outputs a calculated size of one or more recorded features detected in the optical data. For example, the algorithm may be configured to analyze all of the optical data, and identify protrusions from the GI tract into the lumen that have a characteristic shape (e.g., a polyp shape). The size of each identified protrusion is calculated, and the protrusions are grouped by size. For example, the protrusions may be assigned to bins based on accepted clinical size ranges, e.g., to a small bin (less than or equal to 5 mm), a medium bin (between 6 and 9 mm), and a large bin (greater than or equal to 10 mm). For some applications, protrusions having at least a minimum size, and/or assigned to the medium or large bin, are displayed to the physician. Optionally, protrusions having a size lower than the minimum size are also displayed in a separate area of the display, or can be selected by the physician for display. In this manner, the physician is presented with the most-suspicious images first, such that she can immediately identify the patient as requiring a follow up endoscopic procedure.

Alternatively or additionally, the physician reviews all of the optical data acquired during screening of a patient, and identifies (e.g., with a mouse) two points on the screen, which typically surround a suspected pathological entity. The algorithm displays to the physician the absolute distance between the two identified points.

Further alternatively or additionally, the algorithm analyzes the optical data, and places a grid of points on the optical data, each point being separated from an adjacent point by a fixed distance (e.g., 1 cm).

For some applications, the algorithm analyzes the optical data, and the physician evaluates the data subsequently to the screening procedure. Alternatively or additionally, the physician who evaluates the data is located at a site remote from the patient. Further alternatively or additionally, the physician evaluates the data during the procedure, and, for some applications, performs the procedure.

In an embodiment, the optical system comprises a fixed focal length omnidirectional optical system. Fixed focal length optical systems are characterized by providing magnification of a target which increases as the optical system approaches the target. Thus, in the absence of additional information, it is not generally possible to identify the size of a target being viewed through a fixed focal length optical system based strictly on the viewed image.

It is noted that, for some applications, it is desirable to perform techniques described herein in a manner that obtains highly-accurate size assessments (e.g., within 5% or 10% of absolute size). Typical screening procedures, however, do not require this level of accuracy, and still provide useful information to the physician even when size assessments obtained are within 20% to 40% of the correct value (e.g., 30%).

In accordance with an embodiment of the present invention, the size of a target viewed through the fixed focal length optical system is obtained by assuming a reflectivity of the target under constant illumination. Brightness of the target is measured at a plurality of different distances from the optical system, typically at a respective plurality of times. Because measured brightness decreases approximately in proportion to the square of the distance from a source of light and a site where the light is measured, the proportionality constant governing the inverse square relationship can be derived from two or more measurements of the brightness of a particular target. In this manner, the distance between a particular target on the GI tract and the optical system can be determined at one or more points in time. For some applications, the calculation uses as an input thereto a known level of illumination generated by the light source of the optical system.

It is noted that for applications in which the absolute reflectivity of the target is not accurately known, three or more sequential measurements of the brightness of the target are typically performed, and/or at least two temporally closely-spaced sequential measurements are performed. For example, the sequential measurements may be performed during sequential data frames, typically separated by 1/15 second.

Typically, but not necessarily, the brightness of a light source powered by the optical system is adjusted at the time of manufacture and/or automatically during a procedure so as to avoid saturation of the image sensor. For some applications, the brightness of the light source is adjusted separately for a plurality of imaging areas of the optical system.

By measuring the absolute distance to the optical system from each of the targets viewable at one time by the omnidirectional optical system (i.e., a screen of optical data), a two-dimensional or three-dimensional map is generated. This map, in turn, is analyzable by the algorithm to indicate the absolute distance between any two points on the map, because the magnification of the image is derived from the calculated distance to each target and the known focal length. It is noted that this technique provides high redundancy, and that the magnification could be derived from the calculated distance between a single pixel of the image sensor and the target that is imaged on that pixel. The map is input to a feature-identification algorithm, to allow the size of any identified feature (e.g., a polyp) to be determined and displayed to the physician.

Typically, but not necessarily, the image sensor is calibrated at the time of manufacture of the optical system, such that all pixels of the sensor are mapped to ensure that uniform illumination of the pixels produces a uniform output signal. Corrections are typically made for fixed pattern noise (FPN), dark noise, variations of dark noise, and variations in gain. For some applications, each pixel outputs a digital signal ranging from 0-255 that is indicative of brightness.

In an embodiment of the present invention, the size of a protrusion, such as a mid- or large-size polyp, is estimated by: (i) estimating a distance of the protrusion from the optical system, by measuring the brightness of at least (a) a first point on the protrusion relative to the brightness of (b) a second point on the protrusion or on an area of the wall of the GI tract in a vicinity of an edge of the protrusion; (ii) using the estimated distance to calculate a magnification of the protrusion; and (iii) deriving the size based on the magnification. For example, the first point may be in a region of the protrusion that protrudes most from the GI tract wall.

In accordance with another embodiment of the present invention, the size of a target viewed through the fixed focal length optical system is calculated by comparing distortions in magnification of the target when it is imaged, at different times, on different pixels of the optical system. Such distortions may include barrel distortion or pin cushion distortion. Typically, prior to a procedure, or at the time of manufacture of the omnidirectional optical system, at least three reference mappings are performed of a calibrated target at three different known distances from the optical system. The mappings identify relative variations of the magnification across the image plane, and are used as a scaling tool to judge the distance to the object. In optical systems (e.g., in a fixed focal length omnidirectional optical system), distortion of magnification varies non-linearly as a function of the distance of the target to the optical system. Once the distortion is mapped for a number of distances, the observed distortion in magnification of a target imaged in successive data frames during a screening procedure is compared to the data previously obtained for the calibrated target, to facilitate the determination of the size of the target.

By measuring the absolute distance to the optical system from each of the targets viewable at one time by the omnidirectional optical system (i.e., a screen of optical data), a two-dimensional or three-dimensional map is generated, as described hereinabove. This map, in turn, is analyzable by the algorithm to indicate the absolute distance between any two points on the map.

In accordance with yet another embodiment of the present invention, the optical system is configured to have a variable focal length, and the size of a target viewed through the optical system is calculated by imaging the target when the optical system is in respective first and second configurations which cause the system to have respective first and second focal lengths, i.e., to zoom. The first and second configurations differ in that at least one component of the optical system is in a first position along the z-axis of the optical system when the optical system is in the first configuration, and the component is in a second position along the z-axis when the optical system is in the second configuration. For example, the component may comprise a lens of the optical system. Since for a given focal length the magnification of a target is a function of the distance of the target from the optical system, a change in the magnification of the target due to a known change in focal length allows the distance to the object to be determined.

For some applications, a piezoelectric device drives the optical system to switch between the first and second configurations. For example, the piezoelectric device may drive the optical system to switch configurations every 1/15 second, such that successive data frames are acquired in alternating configurations. Typically, but not necessarily, the change in position of the component is less than 1 mm.

By measuring the absolute distance to the optical system from each of the targets viewable at one time by the omnidirectional optical system (i.e., a screen of optical data), a two-dimensional or three-dimensional map is generated, as described hereinabove. This map, in turn, is analyzable by the algorithm to indicate the absolute distance between any two points on the map.

In accordance with still another embodiment of the present invention, the size of a target viewed through the fixed focal length optical system is calculated by projecting a known pattern (e.g., a grid) from the optical system onto the wall of the GI tract. Alternatively, the pattern is projected from a projecting device that is separate from the optical system. The control unit compares a subset of frames of data obtained during a screening procedure (e.g., one frame) to stored calibration data with respect to the pattern in order to determine the distance to the target, and/or to directly determine the size of the target. For example, if the field of view of the optical system includes 100 squares of the grid, then the calibration data may indicate that the optical system is 5 mm from a target at the center of the grid. Alternatively or additionally, it may be determined that each square in the grid is 1 mm wide, allowing the control unit to perform a direct determination of the size of the target. For some applications, the projecting device projects the pattern only during the subset of frames used by the control unit for analyzing the pattern.

In accordance with a further embodiment of the present invention, the size of a target viewed through the fixed focal length optical system is calculated by sweeping one or more lights across the target at a known rate. Typically, but not necessarily, the divergence of the beam of each light is known, and the source(s) of the one or more lights are spaced away from the image sensor, such that the spot size on the GI tract wall indicates the distance to the wall. For some applications, the sweeping of the lights is accomplished using a single beam that is rotated in a circle. For other applications, the sweeping is accomplished by illuminating successive LED's disposed circumferentially around the optical system. For example, 4, 12, or 30 LED's typically at fixed inter-LED angles may be used for this purpose.

For some applications, two non-parallel beams of light are projected generally towards the target from two non-overlapping sources. The angle between the beams may be varied, and when the beams converge while they are on the target, the distance to the target is determined directly, based on the distance between the sources and the known angle. Alternatively or additionally, two or more non-parallel beams (e.g., three or more beams) are projected towards the GI tract wall, and the apparent distance between each of the beams is analyzed to indicate the distance of the optical system from the wall. When performing these calculations, the optical system typically takes into consideration the known geometry of the optical assembly, and the resulting known distortion at different viewing angles.

In accordance with a further embodiment of the present invention, the size of a target viewed through the fixed focal length optical system is calculated by projecting at least one low-divergence light beam, such as a laser beam, onto the target or the GI wall in a vicinity of the target. Because the actual size of the spot produced by the beam on the target or GI wall is known and constant, the spot size as detected by the image sensor indicates the distance to the target or GI wall. For some applications, the optical system projects a plurality of beams in a respective plurality of directions, e.g., between about eight and about 16 directions, such that at least one of the beams is likely to strike any given target of interest, or the GI wall in a vicinity of the target. The optical system typically is configured to automatically identify the relevant spot(s), compare the detected size with the known, actual size, and calculate the distance to the spot(s) based on the comparison. For some applications, the optical system calibrates the calculation using a database of clinical information including detected spot sizes and corresponding actual measured sizes of targets of interest.

In accordance with yet a further embodiment of the present invention, the size of a target viewed through the fixed focal length optical system is determined by comparing the relative size of the target to a scale of known dimensions that is also in the field of view of the image sensor. For example, a portion of the endoscope viewable by the image sensor may have scale markings placed thereupon. In a particular embodiment, the colonoscope comprises a portion thereof that is in direct contact with the wall of the GI tract, and this portion has the scale markings placed thereupon.

In an embodiment, techniques for size determination described hereinabove are utilized during a laparoscopic procedure, e.g., in order to determine the size of an anatomical or pathological feature.

The optical assembly typically comprises an optical member having a rotational shape, at least a distal portion of which is shaped so as to define a curved lateral surface. A distal (forward) end of the optical assembly comprises a convex mirror having a rotational shape that has the same rotation axis as the optical member.

In an embodiment of the present invention, an expert system extracts at least one feature from an acquired image of a protrusion, and compares the feature to a reference library of such features derived from a plurality of images of various protrusions having a range of sizes and distances from the optical system. For example, the at least one feature may include an estimated size of the protrusion. The expert system uses the comparison to categorize the protrusion by size, and, in some embodiments, to generate a suspected diagnosis for use by the physician. For example, the expert system may comprise a neural network, such as a self-learning neural network, which learns to characterize new features from new images by comparing the new images to those stored in the library. For example, images may be classified by size, shape, color, or topography. The expert system typically continuously updates the library.

The optical system is typically configured to enable simultaneous forward and omnidirectional lateral viewing. Light arriving from the forward end of the optical member, and light arriving from the lateral surface of the optical member travel through substantially separate, non-overlapping optical paths. The forward light and the lateral light are typically processed to create two separate images, rather than a unified image. The optical assembly is typically configured to provide different levels of magnification for the forward light and the lateral light. For some applications, the forward view is used primarily for navigation within a body region, while the omnidirectional lateral view is used primarily for inspection of the body region. In these applications, the optically assembly is typically configured such that the magnification of the forward light is less than that of the lateral light.

The optical member is typically shaped so as to define a distal indentation at the distal end of the optical member, i.e., through a central portion of the mirror. A proximal surface of the distal indentation is shaped so as to define a lens that focuses light passing therethrough. In addition, for some applications, the optical member is shaped so as to define a proximal indentation at the proximal end of the optical member. At least a portion of the proximal indentation is shaped so as to define a lens. It is noted that for some applications, the optical member is shaped so as to define a distal protrusion, instead of a distal indentation. Alternatively, the optical member is shaped so as to define a surface (refracting or non-refracting) that is generally flush with the mirror, and which allows light to pass therethrough.

In some embodiments of the present invention, the optical assembly further comprises a distal lens that has the same rotation axis as the optical member. The distal lens focuses light arriving from the forward direction onto the proximal surface of the distal indentation. For some applications, the optical assembly further comprises one or more proximal lenses, e.g., two proximal lenses. The proximal lenses are positioned between the optical member and the image sensor, so as to focus light from the optical member onto the image sensor.

In some embodiments of the present invention, the optical system comprises a light source, which comprises two concentric rings of LEDs encircling the optical member: a side-lighting LED ring and a forward-lighting LED ring. The LEDs of the side-lighting LED ring are oriented such that they illuminate laterally, in order to provide illumination for omnidirectional lateral viewing by the optical system. The LEDs of the forward-lighting LED ring are oriented such that they illuminate in a forward direction, by directing light through the optical member and the distal lens. For some applications, the light source further comprises one or more beam shapers and/or diffusers to narrow or broaden, respectively, the light beams emitted by the LEDs.

Alternatively, the light source comprises a side-lighting LED ring encircling the optical member, and a forward-lighting LED ring positioned in a vicinity of a distal end of the optical member. The LEDs of the forward-lighting LED ring are oriented such that they illuminate in a forward direction. The light source typically provides power to the forward LEDs over at least one power cable, which typically passes along the side of the optical member. For some applications, the power cable is oriented diagonally with respect to a rotation axis of the optical member. Because of movement of the optical system through the lumen, such a diagonal orientation minimizes or eliminates visual interference that otherwise may be caused by the power cable.

In some embodiments of the present invention, the optical system is configured to alternatingly activate the side-lighting and forward-lighting light sources. Image processing circuitry of the endoscope is configured to process forward viewing images only when the forward-viewing light source is illuminated and the side-viewing light source is not illuminated, and to process lateral images only when the side-lighting light source is illuminated and the forward-viewing light source is not illuminated. Such toggling typically reduces any interference that may be caused by reflections caused by the other light source, and/or reduces power consumption and heat generation.

In some embodiments of the present invention, image processing circuitry is configured to capture a series of longitudinally-arranged image segments of an internal wall of a lumen in a subject, while the optical system is moving through the lumen (i.e., being either withdrawn or inserted). The image processing circuitry stitches together individual image segments into a combined continuous image. This image capture and processing technique generally enables higher-magnification imaging than is possible using conventional techniques, ceteris paribus. Using conventional techniques, a relatively wide area must generally be captured simultaneously in order to provide a useful image to the physician. In contrast, the techniques described herein enable the display of such a wide area while only capturing relatively narrow image segments. This enables the optics of the optical system to be focused narrowly on an area of wall having a width approximately equal to that of each image segment.

In some embodiments of the present invention, image processing circuitry produces a stereoscopic image by capturing two images of each point of interest from two respective viewpoints while the optical system is moving, e.g., through a lumen in a subject. For each set of two images, the location of the optical system is determined. Using this location information, the image processing software processes the two images in order to generate a stereoscopic image.

In some embodiments of the present invention, image processing circuitry converts a lateral omnidirectional image of a lumen in a subject to a two-dimensional image. Typically, the image processing circuitry longitudinally cuts the omnidirectional image, and then unrolls the omnidirectional image onto a single plane.

There is therefore provided, in accordance with an embodiment of the invention, apparatus for use in a lumen, including:

a light source, configured to illuminate a vicinity of an object of interest of a wall of the lumen;

an optical system, configured to generate a plurality of images of the vicinity; and

a control unit, configured to:

measure a first brightness of a portion of a first one of the plurality of images generated while the optical system is positioned at a first position with respect to the vicinity,

measure a second brightness of a portion of a second one of the plurality of images generated while the optical system is positioned at a second position with respect to the vicinity, the second position different from the first position, wherein the portion of the second one of the images generally corresponds to the portion of the first one of the images, and

calculate a distance to the vicinity, responsively to the first and second brightnesses.

In an embodiment, the control unit is configured to calculate the distance to the vicinity from a position within the optical system.

In an embodiment, the control unit is configured to calculate the distance to the vicinity from a position a location of which is known with respect to a location of the optical system.

In an embodiment, the vicinity includes a portion of the wall of the lumen adjacent to the object of interest, and wherein the optical system is configured to generate the images of the vicinity which includes the portion of the wall.

In an embodiment, the optical system includes a fixed focal length optical system.

In an embodiment, the control unit is configured to calculate a size of the object of interest responsively to the distance.

In an embodiment, the lumen includes a lumen of a colon of a patient, and wherein the light source is configured to illuminate the vicinity of the object of interest of the wall of the colon.

In an embodiment, the control unit is configured to determine respective locations of the first and second positions with respect to one another, and to calculate the distance at least in part responsively to the respective locations and the first and second brightnesses.

In an embodiment, the control unit is configured to calculate a proportionality constant governing a relationship between the first and second brightnesses, and to calculate the distance using the proportionality constant.

In an embodiment, the control unit is configured to calculate the distance responsively to a known level of illumination of the light source.

In an embodiment, the control unit is configured to calculate the distance responsively to an estimated reflectivity of the vicinity.

In an embodiment, the control unit is configured to calculate the estimated reflectivity responsively to the first and second brightnesses.

In an embodiment, the estimated reflectivity includes a pre-determined estimated reflectivity, and wherein the control unit is configured to calculate the distance responsively to the pre-determined estimated reflectivity of the vicinity.

There is further provided, in accordance with an embodiment of the invention, apparatus for use in a lumen, including:

a light source, configured to illuminate a vicinity of an object of interest of a wall of the lumen;

an optical system, configured to generate an image of the vicinity; and

a control unit, configured to:

assess a distortion of the image, and

calculate a distance to the vicinity responsively to the assessment.

In an embodiment, the control unit is configured to calculate the distance to the vicinity from a position within the optical system.

In an embodiment, the control unit is configured to calculate the distance to the vicinity from a position a location of which is known with respect to a location of the optical system.

In an embodiment, the vicinity includes a portion of the wall of the lumen adjacent to the object of interest, and wherein the optical system is configured to generate the image of the vicinity which includes the portion of the wall.

In an embodiment, the optical system includes a fixed focal length optical system.

In an embodiment, the control unit is configured to calculate a size of the object of interest responsively to the distance.

In an embodiment, the lumen includes a lumen of a colon of a patient, and wherein the light source is configured to illuminate the vicinity of the object of interest of the wall of the colon.

In an Embodiment:

the optical system is configured to generate a plurality of images of the vicinity, and

the control unit is configured to:

assess the distortion by comparing a first distortion of a portion of a first one of the plurality of images generated while the optical system is positioned at a first position, with a second distortion of a portion of a second one of the plurality of images generated while the optical system is positioned at a second position, the second position different from the first position, and the portion of the second one of the images generally corresponding to the portion of the first one of the images, and

calculate the distance responsively to the comparison.

In an embodiment, the optical system includes an image sensor including an array of pixel cells, and wherein a first set of the pixel cells generates the portion of the first one of the images, and a second set of the pixel cells generates the portion of the second one of the images, the first and second sets of the pixel cells located at respective first and second areas of the image sensor, which areas are associated with different distortions.

There is still further provided; in accordance with an embodiment of the invention, apparatus for use in a lumen, including:

a light source, configured to illuminate a vicinity of an object of interest of a wall of the lumen;

an optical system having a variable focal length, the optical system configured to generate an image of the vicinity; and

a control unit, configured to:

set the optical system to have a first focal length, and measure a first magnification of a portion of the image generated while the optical system has the first focal length,

set the optical system to have a second focal length, different from the first focal length, and measure a second magnification of the portion of the image generated while the optical system has the second focal length,

compare the first and second magnifications, and

calculate a distance to the vicinity, responsively to the comparison.

In an embodiment, the control unit is configured to calculate the distance to the vicinity from a position within the optical system.

In an embodiment, the control unit is configured to calculate the distance to the vicinity from a position a location of which is known with respect to a location of the optical system.

In an embodiment, the control unit is configured to calculate the distance responsively to the comparison and a difference between the first and second focal lengths.

In an embodiment, the vicinity includes a portion of the wall of the lumen adjacent to the object of interest, and wherein the optical system is configured to generate the image of the vicinity which includes the portion of the wall.

In an embodiment, the control unit is configured to calculate a size of the object of interest responsively to the distance.

In an embodiment, the lumen includes a lumen of a colon of a patient, and wherein the light source is configured to illuminate the vicinity of the object of interest of the wall of the colon.

In an embodiment, the optical system includes a movable component, a position of which sets the focal length, and wherein the control unit is configured to set the optical system to have the first and second focal lengths by setting the position of the movable component.

In an embodiment, the movable component includes a lens.

In an embodiment, the optical system includes a piezoelectric device configured to set the position of the movable component.

In an embodiment, the control unit is configured to set the position of the movable component such that a change in position of the component between the first and second focal lengths is less than 1 mm.

There is yet further provided, in accordance with an embodiment of the invention, apparatus for use in a lumen, including:

a light source, configured to illuminate a vicinity of an object of interest of a wall of the lumen;

an optical system having a variable focal length, the optical system configured to generate an image of the vicinity; and

a control unit, configured to:

set the optical system to have a first focal length, and drive the optical system to generate a first image of a portion of the vicinity, while the optical system has the first focal length,

set the optical system to have a second focal length, different from the first focal length, and drive the optical system to generate a second image of the portion, while the optical system has the second focal length,

compare respective apparent sizes of the first and second images of the portion generated while the optical system has the first and second focal lengths, respectively, and

calculate a distance to the vicinity, responsively to the comparison.

In an embodiment, the control unit is configured to calculate the distance to the vicinity from a position within the optical system.

In an embodiment, the control unit is configured to calculate the distance to the vicinity from a position a location of which is known with respect to a location of the optical system.

There is also provided, in accordance with an embodiment of the invention, apparatus for use in a lumen, including:

a projecting device, configured to project a projected pattern onto an imaging area within the lumen;

an optical system, configured to generate an image of the imaging area; and

a control unit, configured to:

detect a pattern in the generated image,

analyze the detected pattern, and

responsively to the analysis, calculate a parameter selected from the group consisting of: a distance to a vicinity of an object of interest of the lumen within the imaging area, and a size of the object of interest.

In an embodiment, the control unit is configured to calculate the distance to the vicinity from a position within the optical system.

In an embodiment, the control unit is configured to calculate the distance to the vicinity from a position a location of which is known with respect to a location of the optical system.

In an embodiment, the optical system includes a fixed focal length optical system.

In an embodiment, the control unit is configured to calculate the size of the object of interest responsively to the distance.

In an embodiment, the lumen includes a lumen of a colon of a patient, and wherein the projecting device is configured to project the projected pattern onto the imaging area within the colon.

In an embodiment, the projected pattern includes a grid, and wherein the projecting device is configured to project the grid onto the imaging area.

In an embodiment, the imaging area includes a portion of a wall of the lumen, and wherein the projecting device is configured to project the projected pattern onto the portion of the wall.

In an embodiment, the optical system includes a light source, configured to illuminate the imaging area during the generating of the image, and configured to function as the projecting device during at least a portion of a time period during the generating of the image.

In an embodiment, the control unit is configured to analyze the detected pattern by comparing the detected pattern to calibration data with respect to the projected pattern.

In an Embodiment:

the projected pattern includes a projected grid,

the calibration data includes a property of the projected grid selected from the group consisting of: a number of shapes defined by the projected grid, and a number of intersection points defined by the projected grid, and

the control unit is configured to analyze the detected grid by comparing the selected property of the detected grid with the selected property of the projected grid.

In an Embodiment:

the projected pattern includes a projected grid,

the calibration data includes at least one dimension of shapes defined by the projected grid, and

the control unit is configured to calculate the size of the object of interest responsively to the detected grid and the at least one dimension.

There is additionally provided, in accordance with an embodiment of the invention, apparatus for use in a lumen, including:

a projecting device, configured to project a beam onto an imaging area within the lumen, the beam having a known size at its point of origin, and a known divergence;

an optical system, configured to generate an image of the imaging area; and

a control unit, configured to:

detect a spot of light generated by the beam in the generated image, and

responsively to an apparent size of the spot, the known beam size, and the known divergence, calculate a distance to a vicinity of an object of interest of the lumen within the imaging area.

In an embodiment, the control unit is configured to calculate the distance to the vicinity from a position within the optical system.

In an embodiment, the control unit is configured to calculate the distance to the vicinity from a position a location of which is known with respect to a location of the optical system.

In an embodiment, the beam has a low divergence, and wherein the projecting device is configured to project the low-divergence beam.

In an embodiment, the projecting device includes a laser.

In an embodiment, the optical system includes a fixed focal length optical system.

In an embodiment, the control unit is configured to calculate a size of the object of interest responsively to the distance.

In an embodiment, the lumen includes a lumen of a colon of a patient, and wherein the projecting device is configured to project the beam onto the imaging area within the colon.

In an embodiment, the projecting device is configured to sweep the projected beam across the imaging area.

In an embodiment, the projecting device is configured to sweep the projected beam by illuminating successive light sources disposed around the optical system.

There is yet additionally provided, in accordance with an embodiment of the invention, apparatus for use in a lumen, including:

a projecting device, including two non-overlapping light sources at a known distance from one another, the projecting device configured to project, from the respective light sources, two non-parallel beams at an angle with respect to one another, onto an imaging area within the lumen;

an optical system, configured to generate an image of the imaging area; and

a control unit, configured to:

detect respective spots of light generated by the beams in the generated image, and

responsively to the known distance, an apparent distance between the spots, and the angle, calculate a distance to a vicinity of an object of interest of the lumen within the imaging area.

In an embodiment, the control unit is configured to calculate the distance to the vicinity from a position within the optical system.

In an embodiment, the control unit is configured to calculate the distance to the vicinity from a position a location of which is known with respect to a location of the optical system.

In an embodiment, the optical system includes a fixed focal length optical system.

In an embodiment, the control unit is configured to calculate the size of the object of interest responsively to the distance.

In an embodiment, the lumen includes a lumen of a colon of a patient, and wherein the projecting device is configured to project the beam onto the imaging area within the colon.

In an embodiment, the projecting device is configured to set the angle, and wherein the control unit drives the projecting device to set the angle such that the apparent distance between the spots approaches or reaches zero.

There is still additionally provided, in accordance with an embodiment of the invention, a method for use in a lumen, including:

illuminating a vicinity of an object of interest of a wall of the lumen;

generating a first image and a second image of the vicinity from a first position and a second position, respectively, the second position different from the first position;

measuring a first brightness of a portion of the first image, and a second brightness of a portion of the second image, the portion of the second image generally corresponding to the portion of the first image; and

calculating a distance to the vicinity, responsively to the first and second brightnesses.

In an embodiment, calculating the distance includes calculating the distance to the vicinity from the first position or the second position.

In an embodiment, calculating the distance includes calculating the distance to the vicinity from a third position, a location of which is known with respect to at least one of the first and second positions.

In an embodiment, the vicinity includes a portion of the wall of the lumen adjacent to the object of interest, and wherein generating the first and second images includes generating the first and second images of the vicinity which includes the portion of the wall.

In an embodiment, generating includes generating the first and second images using a fixed focal length optical system.

In an embodiment, the method includes calculating a size of the object of interest responsively to the distance.

In an embodiment, the lumen includes a lumen of a colon of a patient, and wherein illuminating includes illuminating the vicinity of the object of interest of the wall of the colon.

In an embodiment, calculating the distance includes determining respective locations of the first and second positions with respect to one another, and calculating the distance at least in part responsively to the respective locations and the first and second brightnesses.

In an embodiment, calculating the distance includes calculating a proportionality constant governing a relationship between the first and second brightnesses, and calculating the distance using the proportionality constant.

In an embodiment, calculating the distance includes calculating the distance responsively to a known level of illumination of the light source.

In an embodiment, calculating the distance includes calculating the distance responsively to an estimated reflectivity of the vicinity.

In an embodiment, calculating the distance includes calculating the estimated reflectivity responsively to the first and second brightnesses.

In an embodiment, the estimated reflectivity includes a pre-determined estimated reflectivity, and wherein calculating the distance includes calculating the distance responsively to the pre-determined estimated reflectivity of the vicinity.

There is still additionally provided, in accordance with an embodiment of the invention, a method for use in a lumen, including:

illuminating a vicinity of an object of interest of a wall of the lumen;

generating an image of the vicinity;

assessing a distortion of the image; and

calculating a distance to the vicinity responsively to the assessing.

In an embodiment, generating the image includes generating the image from a first position within the lumen, and wherein calculating the distance includes calculating the distance to the vicinity from a second position, a location of which is known with respect to the first position.

In an embodiment, the vicinity includes a portion of the wall of the lumen adjacent to the object of interest, and wherein generating the image includes generating the image of the vicinity which includes the portion of the wall.

In an embodiment, generating the image includes generating the image using a fixed focal length optical system.

In an embodiment, the method includes calculating a size of the object of interest responsively to the distance.

In an embodiment, the lumen includes a lumen of a colon of a patient, and wherein illuminating includes illuminating the vicinity of the object of interest of the wall of the colon.

In an embodiment, generating the image includes generating a plurality of images of the vicinity, and wherein calculating the distance includes:

assessing the distortion by comparing a first distortion of a portion of a first one of the plurality of images generated from a first position, with a second distortion of a portion of a second one of the plurality of images generated from a second position, the second position different from the first position, and the portion of the second one of the images generally corresponding to the portion of the first one of the images; and

calculating the distance responsively to the comparison.

In an embodiment, generating the plurality of images includes:

generating the portion of the first one of the images using a first set of pixel cells of an array of pixel cells of an image sensor; and

generating the portion of the second one of the images using a second set of the pixel cells,

wherein the first and second sets of the pixel cells are located at respective first and second areas of the image sensor, which areas are associated with different distortions.

There is also provided, in accordance with an embodiment of the invention, a method for use in a lumen, including:

inserting an optical system into the lumen;

illuminating a vicinity of an object of interest of a wall of the lumen;

using the optical system, generating a first image of the vicinity while the optical system has a first focal length, and a second image of the vicinity while the optical system has a second focal length, different from the first focal length;

measuring a first magnification of a portion of the first image generated while the optical system has the first focal length, and a second magnification of a portion of the second image generated while the optical system has the second focal length, the portion of the second image generally corresponding to the portion of the first image;

comparing the first and second magnifications; and

calculating a distance to the vicinity, responsively to the comparison.

In an embodiment, calculating the distance includes calculating the distance to the vicinity from a position within the optical system.

In an embodiment, calculating the distance includes calculating the distance to the vicinity from a position a location of which is known with respect to a location of the optical system.

In an embodiment, calculating the distance includes calculating the distance responsively to the comparison and a difference between the first and second focal lengths.

In an embodiment, the vicinity includes a portion of the wall of the lumen adjacent to the object of interest, and wherein generating the image includes generating the image of the vicinity which includes the portion of the wall.

In an embodiment, the method includes calculating a size of the object of interest responsively to the distance.

In an embodiment, the lumen includes a lumen of a colon of a patient, and wherein illuminating includes illuminating the vicinity of the object of interest of the wall of the colon.

There is further provided, in accordance with an embodiment of the invention, a method for use in a lumen, including:

inserting an optical system into the lumen;

illuminating a vicinity of an object of interest of a wall of the lumen;

using the optical system, generating a first image of a portion of the vicinity while the optical system has a first focal length, and a second image of the portion while the optical system has a second focal length;

comparing respective apparent sizes of the first and second images of the portion generated while the optical system has the first and second focal lengths, respectively; and

calculating a distance to the vicinity, responsively to the comparison.

In an embodiment, calculating the distance includes calculating the distance to the vicinity from a position within the optical system.

In an embodiment, calculating the distance includes calculating the distance to the vicinity from a position a location of which is known with respect to a location of the optical system.

There is yet further provided, in accordance with an embodiment of the invention, a method for use in a lumen, including:

projecting a projected pattern onto an imaging area within the lumen;

generating an image of the imaging area;

detecting a pattern in the generated image;

analyzing the detected pattern;

responsively to the analysis, calculating a parameter selected from the group consisting of a distance to a vicinity of an object of interest of the lumen within the imaging area, and a size of the object of interest.

In an embodiment, generating the image includes generating the image from a first position within the lumen, and wherein calculating the distance includes calculating the distance to the vicinity from a second position a location of which is known with respect to the first position.

In an embodiment, generating the image includes generating the image using a fixed focal length optical system.

In an embodiment, the method includes calculating the size of the object of interest responsively to the distance.

In an embodiment, the lumen includes a lumen of a colon of a patient, and wherein projecting includes projecting the projected pattern onto the imaging area within the colon.

In an embodiment, projecting the projected pattern includes projecting a grid onto the imaging area.

In an embodiment, the imaging area includes a portion of a wall of the lumen, and wherein projecting the projected pattern includes projecting the projected pattern onto the portion of the wall.

In an embodiment, generating the image includes illuminating the image area using a light source, and wherein projecting the projected pattern includes projecting the projected pattern using the light source during at least a portion of a time period during the generating of the image.

In an embodiment, analyzing the detected pattern includes comparing the detected pattern to calibration data with respect to the projected pattern.

In an Embodiment:

the projected pattern includes a projected grid,

the calibration data includes a property of the projected grid selected from the group consisting of: a number of shapes defined by the projected grid, and a number of intersection points defined by the projected grid, and

analyzing includes analyzing the detected grid by comparing the selected property of the detected grid with the selected property of the projected grid.

In an Embodiment:

the projected pattern includes a projected grid,

the calibration data include at least one dimension of shapes defined by the projected grid, and

analyzing includes calculating the size of the object of interest responsively to the detected grid and the at least one dimension.

There is still further provided, in accordance with an embodiment of the invention, a method for use in a lumen, including:

projecting a beam onto an imaging area within the lumen, the beam having a known size at its point of origin, and a known divergence;

generating an image of the imaging area;

detecting a spot of light generated by the beam in the generated image; and

responsively to an apparent size of the spot, the known beam size, and the known divergence, calculating a distance to a vicinity of an object of interest of the lumen within the imaging area.

In an embodiment, generating the image includes generating the image from a first position within the lumen, and wherein calculating the distance includes calculating the distance to the vicinity from a second position a location of which is known with respect to the first position.

There is also provided, in accordance with an embodiment of the invention, a method for use in a lumen, including:

projecting, from two non-overlapping positions within the lumen at a known distance from one another, two respective non-parallel beams at an angle with respect to one another, onto an imaging area within the lumen;

generating an image of the imaging area;

detecting respective spots of light generated by the beams in the generated image; and

responsively to the known distance, an apparent distance between the spots, and the angle, calculating a distance to a vicinity of an object of interest of the lumen within the imaging area.

In an embodiment, generating the image includes generating the image from a first position within the lumen, and wherein calculating the distance includes calculating the distance to the vicinity from a second position a location of which is known with respect to the first position.

In an embodiment, generating the image includes generating the image using a fixed focal length optical system.

In an embodiment, the method includes calculating the size of the object of interest responsively to the distance.

In an embodiment, the lumen includes a lumen of a colon of a patient, and wherein projecting includes projecting the beam onto the imaging area within the colon.

In an embodiment, projecting includes setting the angle such that the apparent distance between the spots approaches or reaches zero.

The present invention will be more fully understood from the following detailed description of preferred embodiments thereof, taken together with the drawings, in which:

### BRIEF DESCRIPTION OF THE DRAWINGS

FIG. 1 is a schematic cross-sectional illustration of an optical system for use in an endoscope, in accordance with an embodiment of the present invention;

FIGS. 2A and 2B are schematic cross-sectional illustrations of light passing through the optical system of FIG. 1, in accordance with an embodiment of the present invention;

FIG. 3 is a schematic cross-sectional illustration of a light source for use in an endoscope, in accordance with an embodiment of the present invention; and

FIG. 4 is a schematic cross-sectional illustration of another light source for use in an endoscope, in accordance with an embodiment of the present invention.

### DETAILED DESCRIPTION OF EMBODIMENTS

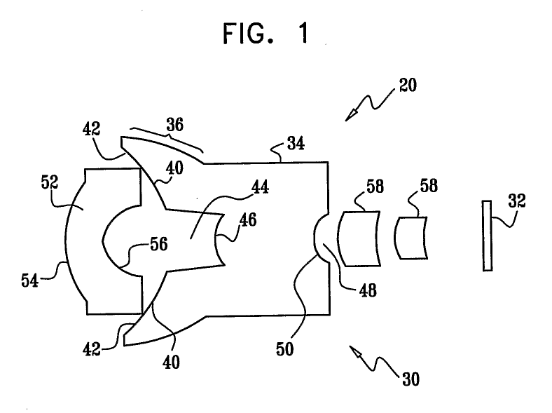

FIG. 1 is a schematic cross-sectional illustration of an optical system 20 for use in an endoscope (e.g., a colonoscope), in accordance with an embodiment of the present invention. Optical system 20 comprises an optical assembly 30 and an image sensor 32, such as a CCD or CMOS sensor. Optical system 20 further comprises mechanical support structures, which, for clarity of illustration, are not shown in the figure. Optical system 20 is typically integrated into the distal end of an endoscope (integration not shown). Optical system 20 further comprises a control unit (not shown), which is configured to carry out the image processing and analysis techniques described hereinbelow, and a light source (not shown), which is configured to illuminate the portion of the lumen being imaged. The control unit is typically positioned externally to the body of the patient, and typically comprises a standard personal computer or server with appropriate memory, communication interfaces and software for carrying out the functions prescribed by relevant embodiments of the present invention. This software may be downloaded to the control unit in electronic form over a network, for example, or it may alternatively be supplied on tangible media, such as CD-ROM. Alternatively, all or a portion of the control unit is positioned on or in a portion of the endoscope that is inserted into the patient's body.

Optical assembly 30 comprises an optical member 34 having a rotational shape. Typically, at least a distal portion 36 of the optical member is shaped so as to define a curved lateral surface, e.g., a hyperbolic, parabolic, ellipsoidal, conical, or semi-spherical surface. Optical member 34 comprises a transparent material, such as acrylic resin, polycarbonate, or glass. For some applications, all or a portion of the lateral surface of optical member 34 other than portion 36 is generally opaque, in order to prevent unwanted light from entering the optical member.

Optical assembly 30 further comprises, at a distal end thereof, a convex mirror 40 having a rotational shape that has the same rotation axis as optical member 34. Mirror 40 is typically aspheric, e.g., hyperbolic or conical. Alternatively, mirror 40 is semi-spherical. Mirror 40 is typically formed by coating a forward-facing concave portion 42 of optical member 34 with a non-transparent reflective coating, e.g., aluminum, silver, platinum, a nickel-chromium alloy, or gold. Such coating may be performed, for example, using vapor deposition, sputtering, or plating. Alternatively, mirror 40 is formed as a separate element having the same shape as concave portion 42, and the mirror is subsequently coupled to optical member 34.

Optical member 34 is typically shaped so as to define a distal indentation 44 at the distal end of the optical member, i.e., through a central portion of mirror 40. Distal indentation 44 typically has the same rotation axis as optical member 34. A proximal surface 46 of distal indentation 44 is shaped so as to define a lens that focuses light passing therethrough. Alternatively, proximal surface 46 is non-focusing. For some applications, optical member 34 is shaped so as to define a distally-facing protrusion from mirror 40. Alternatively, optical member 34 is shaped without indentation 44, but instead mirror 40 includes a non-mirrored portion in the center thereof.

For some applications, optical member 34 is shaped so as to define a proximal indentation 48 at the proximal end of the optical member. Proximal indentation 48 typically has the same rotation axis as optical member 34. At least a portion of proximal indentation 48 is shaped so as to define a lens 50. For some applications, lens 50 is aspheric.

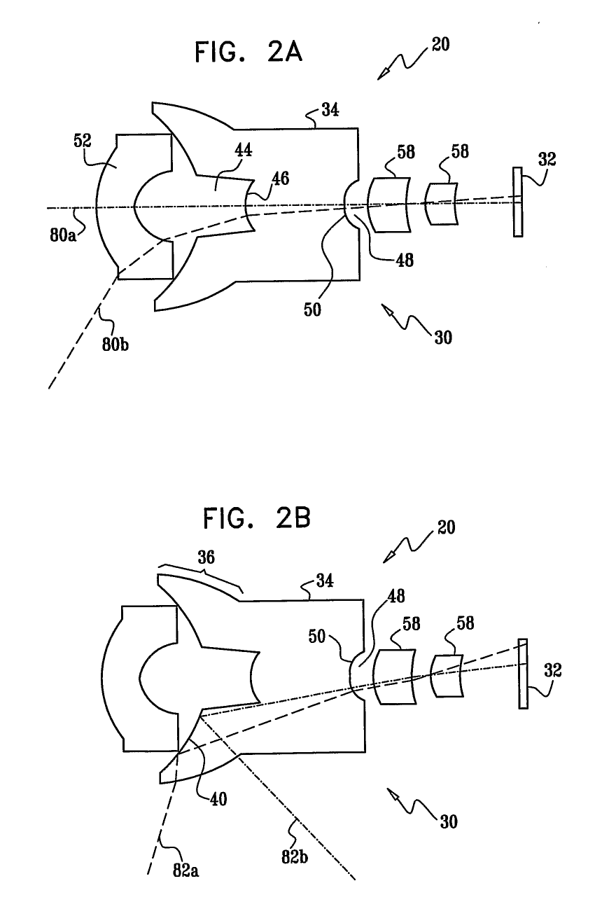

In an embodiment of the present invention, optical assembly 30 further comprises a distal lens 52 that has the same rotation axis as optical member 34. Distal lens 52 focuses light arriving from the forward (proximal) direction onto proximal surface 46 of distal indentation 44, as described hereinbelow with reference to FIG. 2A. For some applications, distal lens 52 is shaped so as to define a distal convex aspheric surface 54, and a proximal concave aspheric surface 56. Typically, the radius of curvature of proximal surface 56 is less than that of distal surface 54. Distal lens 52 typically comprises a transparent optical plastic material such as acrylic resin or polycarbonate, or it may comprise glass.

For some applications, optical assembly 30 further comprises one or more proximal lenses 58, e.g., two proximal lenses 58. Proximal lenses 58 are positioned between optical member 34 and image sensor 32, so as to focus light from the optical member onto the image sensor. Typically, lenses 58 are aspheric, and comprise a transparent optical plastic material, such as acrylic resin or polycarbonate, or they may comprise, for example, glass, an alicyclic acrylate, a cycloolefin polymer, or polysulfone.

Reference is now made to FIGS. 2A and 2B, which are schematic cross-sectional illustrations of light passing through optical system 20, in accordance with an embodiment of the present invention. Optical system 20 is configured to enable simultaneous forward and omnidirectional lateral viewing. As shown in FIG. 2A, forward light, symbolically represented as lines 80a and 80b, enters optical assembly 30 distal to the assembly. Typically, the light passes through distal lens 52, which focuses the light onto proximal surface 46 of distal indentation 44. Proximal surface 46 in turn focuses the light onto lens 50 of proximal indentation 48, which typically further focuses the light onto proximal lenses 58. The proximal lenses still further focus the light onto image sensor 32, typically onto a central portion of the image sensor.

As shown in FIG. 2B, lateral light, symbolically represented as lines 82a and 82b, laterally enters optical assembly 30. The light is refracted by distal portion 36 of optical member 34, and then reflected by mirror 40. The light then passes through lens 50 of proximal indentation 48, which typically further focuses the light onto proximal lenses 58. The proximal lenses still further focus the light onto image sensor 32, typically onto a peripheral portion of the image sensor.

As can be seen, the forward light and the lateral light travel through substantially separate, non-overlapping optical paths. The forward light and the lateral light are typically processed to create two separate images, rather than a unified image. Optical assembly 30 is typically configured to provide different levels of magnification for the forward light and the lateral light. The magnification of the forward light is typically determined by configuring the shape of distal lens 52, proximal surface 46, and the central region of lens 50 of proximal indentation 48. On the other hand, the magnification of the lateral light is typically determined by configuring the shape of distal portion 36 of optical member 34 and the peripheral region of lens 50 of proximal indentation 48.

For some applications, the forward view is used primarily for navigation within a body region, while the omnidirectional lateral view is used primarily for inspection of the body region. In these applications, optically assembly 30 is typically configured such that the magnification of the forward light is less than that of the lateral light.

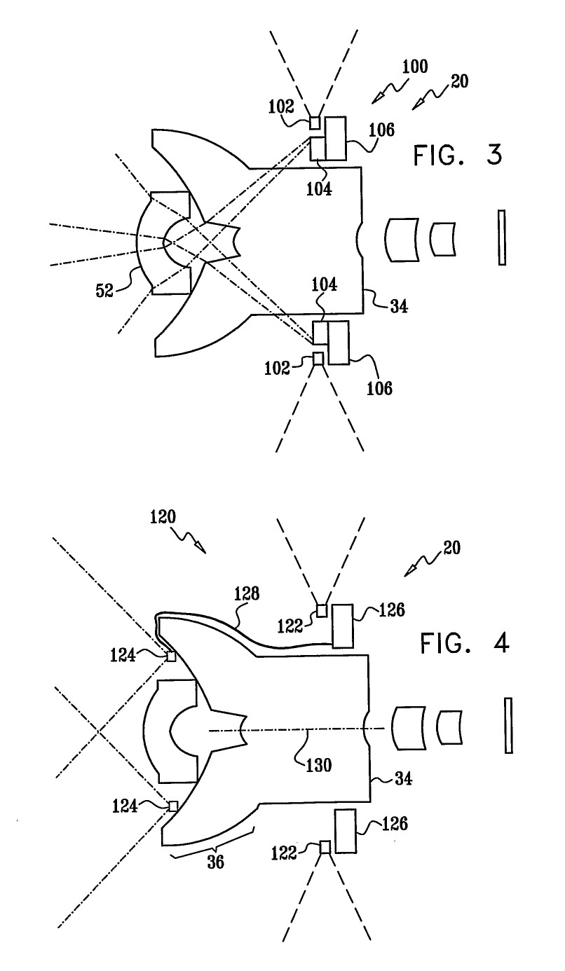

Reference is now made to FIG. 3, which is a schematic cross-sectional illustration of a light source 100 for use in an endoscope, in accordance with an embodiment of the present invention. Although light source 100 is shown and described herein as being used with optical system 20, the light source may also be used with other endoscopic optical systems that provide both forward and lateral viewing.

Light source 100 comprises two concentric rings of LEDs encircling optical member 34: a side-lighting LED ring 102 and a forward-lighting LED ring 104. Each of the rings typically comprises between about 4 and about 12 individual LEDs. The LEDs are typically supported by a common annular support structure 106. Alternatively, the LEDs of each ring are supported by separate support structures, or are supported by optical member 34 (configurations not shown). Alternatively or additionally, light source 100 comprises one or more LEDs (or other lights) located at a different site, but coupled to support structure 106 via optical fibers (configuration not shown). It is thus to be appreciated that embodiments described herein with respect to LEDs directly illuminating an area could be modified, mutatis mutandis, such that light is generated at a remote site and conveyed by optical fibers. As appropriate for various applications, suitable remote sites may include a site near the image sensor, a site along the length of the endoscope, or a site external to the lumen.

The LEDs of side-lighting LED ring 102 are oriented such that they illuminate laterally, in order to provide illumination for omnidirectional lateral viewing by optical system 20. The LEDs of forward-lighting LED ring 104 are oriented such that they illuminate in a forward direction, by directing light through optical member 34 and distal lens 52. Typically, as shown in FIG. 3, side-lighting LED ring 102 is positioned further from optical member 34 than is forward-lighting LED ring 104. Alternatively, the side-lighting LED ring is positioned closer to optical member 34 than is the forward-lighting LED ring. For example, the LEDs of the rings may be positioned such that the LEDs of the forward-lighting LED ring do not block light emitted from the LEDs of the side-lighting LED ring, or the side-lighting LED ring may be placed distal or proximal to the forward-lighting LED ring (configurations not shown).

For some applications, light source 100 further comprises one or more beam shapers and/or diffusers to narrow or broaden, respectively, the light beams emitted by the LEDs. For example, beam shapers may be provided to narrow the light beams emitted by the LEDs of forward-lighting LED ring 104, and/or diffusers may be provided to broaden the light beams emitted by the LEDs of side-lighting LED ring 102.

Reference is now made to FIG. 4, which is a schematic cross-sectional illustration of a light source 120 for use in an endoscope, in accordance with an embodiment of the present invention. Although light source 120 is shown and described as being used with optical system 20, the light source may also be used with other endoscopic optical systems that provide both forward and lateral viewing.

Light source 120 comprises a side-lighting LED ring 122 encircling optical member 34, and a forward-lighting LED ring 124 positioned in a vicinity of a distal end of optical member 34. Each of the rings typically comprises between about 4 and about 12 individual LEDs. The LEDs of side-lighting LED ring 122 are oriented such that they illuminate laterally, in order to provide illumination for omnidirectional lateral viewing by optical system 20. The LEDs of side-lighting LED ring 122 are typically supported by an annular support structure 126, or by optical member 34 (configuration not shown).

The LEDs of forward-lighting LED ring 124 are oriented such that they illuminate in a forward direction. The LEDs of forward-lighting LED ring 124 are typically supported by optical member 34. Light source 120 typically provides power to the LEDs over at least one power cable 128, which typically passes along the side of optical member 34. (For some applications, power cable 128 is flush with the side of optical member 34.) In an embodiment, power cable 128 is oriented diagonally with respect to a rotation axis 130 of optical member 34, as the cable passes distal portion 36. (In other words, if power cable 128 passes the proximal end of distal portion 36 at “12 o'clock,” then it may pass the distal end of distal portion 36 at “2 o'clock.”) As described hereinbelow, such a diagonal orientation minimizes or eliminates visual interference that otherwise may be caused by the power cable.

For some applications, light source 120 further comprises one or more beam shapers and/or diffusers to narrow or broaden, respectively, the light beams generated by the LEDs. For example, diffusers may be provided to broaden the light beams generated by the LEDs of side-lighting LED ring 122 and/or forward-lighting LED ring 124.

Although light source 100 (FIG. 3) and light source 120 (FIG. 4) are described herein as comprising LEDs, the light sources may alternatively or additionally comprise other illuminating elements. For example, the light sources may comprise optical fibers illuminated by a remote light source, e.g., external to the endoscope or in the handle of the endoscope.

In an embodiment of the present invention, optical system 20 comprises a side-lighting light source and a forward-lighting light source. For example, the side-lighting light source may comprise side-lighting LED ring 102 or side-lighting LED ring 122, or any other side-lighting light source known in the art. Similarly, the forward-lighting light source may comprise forward-lighting LED ring 104 or forward-lighting LED ring 124, or any other forward-lighting light source known in the art. Optical system 20 is configured to alternatingly activate the side-lighting and forward-lighting light sources, typically at between about 10 and about 20 Hz, although faster or slower rates may be appropriate depending on the desired temporal resolution of the imaging data.

For some applications, only one of the light sources is activated for a desired length of time (e.g., greater than one minute), and video data are displayed based on the images illuminated by that light source. For example, the forward-lighting light source may be activated during initial advancement of a colonoscope to a site slightly beyond a target site of interest, and the side-lighting light source may be activated during slow retraction of the colonoscope, in order to facilitate close examination of the target site.

Image processing circuitry of the endoscope is configured to process forward-viewing images that were sensed by image sensor 32 during activation of the forward-viewing light source, when the side-viewing light source was not activated. The image processing circuitry is configured to process lateral images that were sensed by image sensor 32 during activation of the side-lighting light source, when the forward-viewing light source was not activated. Such toggling reduces any interference that may be caused by reflections caused by the other light source, and/or reduces power consumption and heat generation. For some applications, such toggling enables optical system 20 to be configured to utilize at least a portion of image sensor 32 for both forward and side viewing.

In an embodiment, a duty cycle is provided to regulate the toggling. For example, the lateral images may be sampled for a greater amount of time than the forward-viewing images (e.g., at time ratios of 1.5:1, or 3:1). Alternatively, the lateral images may be sampled for a lesser amount of time than the forward-viewing images.

In an embodiment, in order to reduce a possible sensation of image flickering due to the toggling, each successive lateral image is continuously displayed until the next lateral image is displayed, and, correspondingly, each successive forward-viewing image is continuously displayed until the next forward-viewing image is displayed. (The lateral and forward-viewing images are displayed on different portions of a monitor.) Thus, for example, even though the sampled forward-viewing image data may include a large amount of dark video frames (because forward illumination is alternated with lateral illumination), substantially no dark frames are displayed.

In an embodiment of the present invention, optical system 20 is configured for use as a gastrointestinal (GI) tract screening device, e.g., to facilitate identification of patients having a GI tract cancer or at risk for same. Although for some applications the endoscope may comprise an element that actively interacts with tissue of the GI tract (e.g., by cutting or ablating tissue), typical screening embodiments of the invention do not provide such active interaction with the tissue. Instead, the screening embodiments typically comprise passing an endoscope through the GI tract and recording data about the GI tract while the endoscope is being passed therethrough. (Typically, but not necessarily, the data are recorded while the endoscope is being withdrawn from the GI tract.) The data are analyzed, and a subsequent procedure is performed to actively interact with tissue if a physician or algorithm determines that this is appropriate.

It is noted that screening procedures using an endoscope are described by way of illustration and not limitation. The scope of the present invention includes performing the screening procedures using an ingestible capsule, as is known in the art. It is also noted that although omnidirectional imaging during a screening procedure is described herein, the scope of the present invention includes the use of non-omnidirectional imaging during a screening procedure.

For some applications, a screening procedure is provided in which optical data of the GI tract are recorded, and an algorithm analyzes the optical data and outputs a calculated size of one or more recorded features detected in the optical data. For example, the algorithm may be configured to analyze all of the optical data, and identify protrusions from the GI tract into the lumen that have a characteristic shape (e.g., a polyp shape). The size of each identified protrusion is calculated, and the protrusions are grouped by size. For example, the protrusions may be assigned to bins based on accepted clinical size ranges, e.g., a small bin (less than or equal to 5 mm), a medium bin (between 6 and 9 mm), and a large bin (greater than or equal to 10 mm). For some applications, protrusions having at least a minimum size, and/or assigned to the medium or large bin, are displayed to the physician. Optionally, protrusions having a size lower than the minimum size are also displayed in a separate area of the display, or can be selected by the physician for display. In this manner, the physician is presented with the most-suspicious images first, such that she can immediately identify the patient as requiring a follow up endoscopic procedure.

Alternatively or additionally, the physician reviews all of the optical data acquired during screening of a patient, and identifies (e.g., with a mouse) two points on the screen, which typically surround a suspected pathological entity. The algorithm displays to the physician the absolute distance between the two identified points.

Further alternatively or additionally, the algorithm analyzes the optical data, and places a grid of points on the optical data, each point being separated from an adjacent point by a fixed distance (e.g., 1 cm).

For some applications, the algorithm analyzes the optical data, and the physician evaluates the data subsequently to the screening procedure. Alternatively or additionally, the physician who evaluates the data is located at a site remote from the patient. Further alternatively or additionally, the physician evaluates the data during the procedure, and, for some applications, performs the procedure.

In an embodiment of the present invention, optical system 20 comprises a fixed focal length omnidirectional optical system, such as described hereinabove with reference to FIGS. 1 and 2. Fixed focal length optical systems are characterized by providing magnification of a target which increases as the optical system approaches the target. Thus, in the absence of additional information, it is not generally possible to identify the size of a target being viewed through a fixed focal length optical system based strictly on the viewed image.

In accordance with an embodiment of the present invention, the control unit obtains the size of a target viewed through the fixed focal length optical system, by measuring brightness of a vicinity of the target while optical system 20 is positioned at a plurality of different positions with respect to the target, each of which has a respective different distance to the target. Because measured brightness decreases approximately in proportion to the square of the distance from a source of light and a site where the light is measured, the proportionality constant governing the inverse square relationship can be derived from two or more measurements of the brightness of a particular target. In this manner, the distance between the vicinity of a particular target on the GI tract and optical system 20 can be determined at one or more points in time. For some applications, the vicinity of the target includes a portion of a wall of the GI tract adjacent to the target. For some applications, the calculation uses as an input thereto a known level of illumination generated by the light source of the optical system, and/or an estimated or assumed reflectivity of the target and/or the GI tract wall.

In order to perform this calculation, the control unit typically determines the absolute or relative locations of optical system 20 at each of the different positions. For example, the control unit may use one or more position sensors, as is known in the art of medical position sensing. Alternatively, for applications in which the positions are longitudinally arranged with the GI tract (such as when the optical system is positioned at the plurality of positions by advancing or withdrawing the endoscope through the GI tract), the control unit determines the locations of the positions with respect to one another by detecting the motion of the optical system, such as by sensing markers on an elongate carrier which is used to advance and withdraw the optical system.

It is noted that for applications in which the absolute reflectivity of the target is not accurately known, three or more sequential measurements of the brightness of the target are typically performed, and/or at least two temporally closely-spaced sequential measurements are performed. For example, the sequential measurements may be performed during sequential data frames, typically separated by 1/15 second. In this manner, relative geometrical orientations of the various aspects of the observed image, the light source, and the image sensor, are generally maintained.

Typically, but not necessarily, the brightness of a light source powered by the optical system is adjusted at the time of manufacture and/or automatically during a procedure so as to avoid saturation of the image sensor. For some applications, the brightness of the light source is adjusted separately for a plurality of imaging areas of the optical system.

By measuring the absolute distance to the optical system from each of the targets viewable at one time by the omnidirectional optical system (i.e., a screen of optical data), the control unit generates a two-dimensional or three-dimensional map. This map, in turn, is analyzable by the algorithm to indicate the absolute distance between any two points on the map, because the magnification of the image is derived from the calculated distance to each target and the known focal length. It is noted that this technique provides high redundancy, and that the magnification could be derived from the calculated distance between a single pixel of the image sensor and the target that is imaged on that pixel. The map is input to a feature-identification algorithm, to allow the size of any identified feature (e.g., a polyp) to be determined and displayed to the physician.

Typically, but not necessarily, image sensor 32 is calibrated at the time of manufacture of optical system 20, such that all pixels of the sensor are mapped to ensure that uniform illumination of the pixels produces a uniform output signal. Corrections are typically made for fixed pattern noise (FPN), dark noise, variations of dark noise, and variations in gain. For some applications, each pixel outputs a digital signal ranging from 0-255 that is indicative of brightness.

In an embodiment of the present invention, the control unit estimates the size of a protrusion, such as a mid- or large-size polyp, by: (i) estimating a distance of the protrusion from the optical system, by measuring the brightness of at least (a) a first point on the protrusion relative to the brightness of (b) a second point on the protrusion or on an area of the wall of the GI tract in a vicinity of an edge of the protrusion; (ii) using the estimated distance to calculate a magnification of the protrusion; and (iii) deriving the size based on the magnification. For example, the first point may be in a region of the protrusion that most protrudes from the GI tract wall. Alternatively or additionally, techniques described herein or known in the art for assessing distance based on brightness are used to determine the distance from the two points, and to estimate the size of the protrusion accordingly.

In an embodiment of the present invention, the control unit calculates the size of a target viewed through fixed focal length optical system 20 by comparing distortions in magnification of the target when it is imaged, at different times, on different pixels of optical system 20. Such distortions may include barrel distortion or pin cushion distortion. Typically, prior to a procedure, or at the time of manufacture of the omnidirectional optical system, at least three reference mappings are performed of a calibrated target at three different known distances from the optical system. The mappings identify relative variations of the magnification across the image plane, and are used as a scaling tool to judge the distance to the object. In optical systems (e.g., in a fixed focal length omnidirectional optical system), distortion of magnification varies non-linearly as a function of the distance of the target to the optical system. Once the distortion is mapped for a number of distances, the observed distortion in magnification of a target imaged in successive data frames during a screening procedure is compared to the data previously obtained for the calibrated target, to facilitate the determination of the size of the target.

By measuring the absolute distance to the optical system from each of the targets viewable at one time by the omnidirectional optical system (i.e., a screen of optical data), the control unit generates a two-dimensional or three-dimensional map, as described hereinabove. This map, in turn, is analyzable by the algorithm to indicate the absolute distance between any two points on the map.

In an embodiment of the present invention, optical system 20 is configured to have a variable focal length, and the control unit calculates the size of a target viewed through optical system 20 by imaging the target when optical system 20 is in respective first and second configurations which cause the system to have respective first and second focal lengths. The first and second configurations differ in that at least one component of optical system 20 is in a first position along the z-axis of the optical system when optical system 20 is in the first configuration, and the component is in a second position along the z-axis when optical system 20 is in the second configuration. For example, the component may comprise a lens of optical system 20. Since for a given focal length the magnification of a target is a function of the distance of the target from the optical system, a change in the magnification of the target due to a known change in focal length allows the distance to the object to be determined.

For some applications, a piezoelectric device drives optical system 20 to switch between the first and second configurations. For some applications, the control unit drives the optical system to switch configurations every 1/15 second, such that successive data frames are acquired in alternating configurations. Typically, but not necessarily, the change in position of the component is less than 1 mm.

By measuring the absolute distance to the optical system from each of the targets viewable at one time by the omnidirectional optical system (i.e., a screen of optical data), the control unit generates a two-dimensional or three-dimensional map, as described hereinabove. This map, in turn, is analyzable by the algorithm to indicate the absolute distance between any two points on the map.

In accordance with still another embodiment of the present invention, the size of a target viewed through the fixed focal length optical system is calculated by projecting a known pattern (e.g., a grid) from the optical system onto the wall of the GI tract. Alternatively, the pattern is projected from a projecting device that is separate from the optical system. A subset of frames of data obtained during a screening procedure (e.g., one frame) is compared to stored calibration data with respect to the pattern in order to determine the distance to the target, and/or to directly determine the size of the target. For some applications, the calibration data includes a property of the grid, such as a number of shapes, such as polygons (e.g., rectangles, such as squares) or circles, defined by the grid, or a number of intersection points defined by the grid. For example, if the field of view of the optical system includes 100 squares of the grid, then the calibration data may indicate that the optical system is 5 mm from a target at the center of the grid. Alternatively or additionally, it may be determined that each square in the grid is 1 mm wide, allowing a direct determination of the size of the target to be performed.

In accordance with a further embodiment of the present invention, the control unit calculates the size of a target viewed through the fixed focal length optical system by driving a projecting device to project a beam onto an imaging area within the GI tract, the beam having a known size at its point of origin, and a known divergence. The control unit detects the spot of light generated by the beam in the generated image, and, responsively to an apparent size of the spot, the known beam size, and the known beam divergence, calculates a distance between the optical system and a vicinity of an object of interest of the GI tract within the imaging area.

For some applications, the control unit is configured to sweep one or more lights across the target at a known rate. For some applications, the projecting device accomplishes the sweeping of the lights using a single beam that is rotated in a circle. For other applications, the sweeping is accomplished by illuminating successive light sources (e.g., LEDs) disposed circumferentially around the optical system. For example, the projecting device may comprise 4-12, or 12-30 light sources typically at fixed inter-light-source angles.

For some applications, a projecting device comprises two non-overlapping sources at a known distance from one another. The projecting device projects, from the respective light sources, two non-parallel beams of light at an angle with respect to one another, generally towards the target. For some applications, the control unit drives the projecting device to vary the angle between the beams, and when the beams converge while they are on the target, the control unit determines the distance to the target directly, based on the distance between the sources and the known angle. Alternatively or additionally, the projecting device projects two or more non-parallel beams (e.g., three or more beams) towards the GI tract wall, and the control unit analyzes the apparent distance between each of the beams to indicate the distance of the optical system from the wall. When performing these calculations, the optical system typically takes into consideration the known geometry of the optical assembly, and the resulting known distortion at different viewing angles.

In accordance with a further embodiment of the present invention, the size of a target viewed through the fixed focal length optical system is calculated by projecting at least one low-divergence light beam, such as a laser beam, onto the target or the GI wall in a vicinity of the target. Because the actual size of the spot produced by the beam on the target or GI wall is known and constant, the spot size as detected by the image sensor indicates the distance to the target or GI wall. For some applications, the optical system projects a plurality of beams in a respective plurality of directions, e.g., between about eight and about 16 directions, such that at least one of the beams is likely to strike any given target of interest, or the GI wall in a vicinity of the target. The optical system typically is configured to automatically identify the relevant spot(s), compare the detected size with the known, actual size, and calculate the distance to the spot(s) based on the comparison. For some applications, the optical system calibrates the calculation using a database of clinical information including detected spot sizes and corresponding actual measured sizes of targets of interest. For some applications, the laser is located remotely from optical assembly 30, and transmits the laser beam via an optical fiber. For example, the laser may be located in an external handle of the endoscope.

In accordance with an embodiment of the present invention, the size of a target viewed through the fixed focal length optical system is determined by comparing the relative size of the target to a scale of known dimensions that is also in the field of view of the image sensor. For example, a portion of the endoscope viewable by the image sensor may have scale markings placed thereupon. In a particular embodiment, the colonoscope comprises a portion thereof that is in direct contact with the wall of the GI tract, and this portion has the scale markings placed thereupon.

In an embodiment, techniques for size determination described hereinabove are utilized during a laparoscopic procedure, e.g., in order to determine the size of an anatomical or pathological feature.

The optical assembly typically comprises an optical member having a rotational shape, at least a distal portion of which is shaped so as to define a curved lateral surface. A distal (forward) end of the optical assembly comprises a convex mirror having a rotational shape that has the same rotation axis as the optical member. (The mirror is labeled “convex” because, as described hereinbelow with reference to the figures, a convex surface of the mirror reflects light striking the mirror, thereby directing the light towards the image sensor.)

In an embodiment of the present invention, an expert system extracts at least one feature from an acquired image of a protrusion, and compares the feature to a reference library of such features derived from a plurality of images of various protrusions having a range of sizes and distances from the optical system. For example, the at least one feature may include an estimated size of the protrusion. The expert system uses the comparison to categorize the protrusion, and to generate a suspected diagnosis for use by the physician.

A number of embodiments of the present invention described herein include techniques for calculating a distance from the optical system to an imaged area or target of interest. It is to be understood that such a distance may be calculated to the imaged area or target from various elements of the optical system, such as an imaging sensor thereof, a surface of an optical component thereof (e.g., a lens thereof), a location within an optical component thereof, or any other convenient location. Alternatively or additionally, such a distance may be calculated from a position outside of the optical system, a location of which position is known with respect to a location of the optical system. Mathematically equivalent techniques for calculating such a distance from arbitrary positions will be evident to those skilled in the art who have read the present application, and are within the scope of the present invention.

Although embodiments of the present invention have been described with respect to medical endoscopes, the techniques described herein are also applicable to other endoscopic applications, such as industrial endoscopy (e.g., pipe inspection).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-13. (canceled)

14. Apparatus for use in a lumen, comprising:
a light source, configured to illuminate a vicinity of an object of interest of a wall of the lumen;
an optical system, configured to enable forward and omnidirectional lateral viewing and to generate an image of the vicinity; and
a control unit, configured to:
assess a distortion of the image, and
calculate a distance to the vicinity responsively to the assessment.

15. The apparatus according to claim 14, wherein the control unit is configured to calculate the distance to the vicinity from a position within the optical system.

16-17. (canceled)

18. The apparatus according to claim 14, wherein the optical system comprises a fixed focal length optical system.

19. The apparatus according to claim 14, wherein the control unit is configured to calculate a size of the object of interest responsively to the distance.

20. (canceled)

21. The apparatus according to claim 14,
wherein the optical system is configured to generate a plurality of images of the vicinity, and
wherein the control unit is configured to:
assess the distortion by comparing a first distortion of a portion of a first one of the plurality of images generated while the optical system is positioned at a first position, with a second distortion of a portion of a second one of the plurality of images generated while the optical system is positioned at a second position, the second position different from the first position, and the portion of the second one of the images generally corresponding to the portion of the first one of the images, and
calculate the distance responsively to the comparison.

22. The apparatus according to claim 21, wherein the optical system comprises an image sensor comprising an array of pixel cells, and wherein a first set of the pixel cells generates the portion of the first one of the images, and a second set of the pixel cells generates the portion of the second one of the images, the first and second sets of the pixel cells located at respective first and second areas of the image sensor, which areas are associated with different distortions.

23. Apparatus for use in a lumen, comprising:
a light source, configured to illuminate a vicinity of an object of interest of a wall of the lumen;
an optical system having a variable focal length, the optical system configured to enable forward and omnidirectional lateral viewing and to generate an image of the vicinity; and
a control unit, configured to:
set the optical system to have a first focal length, and measure a first magnification of a portion of the image generated while the optical system has the first focal length,
set the optical system to have a second focal length, different from the first focal length, and measure a second magnification of the portion of the image generated while the optical system has the second focal length,
compare the first and second magnifications, and
calculate a distance to the vicinity, responsively to the comparison.

24-25. (canceled)

26. The apparatus according to claim 23, wherein the control unit is configured to calculate the distance responsively to the comparison and a difference between the first and second focal lengths.

27-28. (canceled)

29. The apparatus according to claim 23, wherein the lumen includes a lumen of a colon of a patient, and wherein the light source is configured to illuminate the vicinity of the object of interest of the wall of the colon.

30-33. (canceled)

34. Apparatus for use in a lumen, comprising:
a light source, configured to illuminate a vicinity of an object of interest of a wall of the lumen;
an optical system having a variable focal length, the optical system configured to enable forward and omnidirectional lateral viewing and to generate an image of the vicinity; and
a control unit, configured to:
set the optical system to have a first focal length, and drive the optical system to generate a first image of a portion of the vicinity, while the optical system has the first focal length,
set the optical system to have a second focal length, different from the first focal length, and drive the optical system to generate a second image of the portion, while the optical system has the second focal length,
compare respective apparent sizes of the first and second images of the portion generated while the optical system has the first and second focal lengths, respectively, and
calculate a distance to the vicinity, responsively to the comparison.

35. The apparatus according to claim 34, wherein the control unit is configured to calculate the distance to the vicinity from a position within the optical system.

36. The apparatus according to claim 34, wherein the control unit is configured to calculate the distance to the vicinity from a position a location of which is known with respect to a location of the optical system.

37. Apparatus for use in a lumen, comprising:
a projecting device, configured to project a projected pattern onto an imaging area within the lumen;
an optical system, configured to enable forward and omnidirectional lateral viewing and to generate an image of the imaging area; and
a control unit, configured to:
detect a pattern in the generated image,
analyze the detected pattern, and
responsively to the analysis, calculate a parameter selected from the group consisting of: a distance to a vicinity of an object of interest of the lumen within the imaging area, and a size of the object of interest.

38-39. (canceled)

40. The apparatus according to claim 37, wherein the optical system comprises a fixed focal length optical system.

41. The apparatus according to claim 37, wherein the control unit is configured to calculate the size of the object of interest responsively to the distance.

42. The apparatus according to claim 37, wherein the lumen includes a lumen of a colon of a patient, and wherein the projecting device is configured to project the projected pattern onto the imaging area within the colon.

43-45. (canceled)

46. The apparatus according to claim 37, wherein the control unit is configured to analyze the detected pattern by comparing the detected pattern to calibration data with respect to the projected pattern.

47-48. (canceled)

49. Apparatus for use in a lumen, comprising:
a projecting device, configured to project a beam onto an imaging area within the lumen, the beam having a known size at its point of origin, and a known divergence;
an optical system, configured to enable forward and omnidirectional lateral viewing and to generate an image of the imaging area; and
a control unit, configured to:
detect a spot of light generated by the beam in the generated image, and
responsively to an apparent size of the spot, the known beam size, and the known divergence, calculate a distance to a vicinity of an object of interest of the lumen within the imaging area.

50-51. (canceled)

52. The apparatus according to claim 49, wherein the beam has a low divergence, and wherein the projecting device is configured to project the low-divergence beam.

53. (canceled)

54. The apparatus according to claim 49, wherein the optical system comprises a fixed focal length optical system.

55-115. (canceled)

116. Apparatus for use in a lumen, comprising:
a light source, configured to illuminate a vicinity of an object of interest of a wall of the lumen;
an optical system, configured to enable forward and omnidirectional lateral viewing and to generate a plurality of images of the vicinity; and
a control unit, configured to:
measure a first brightness of a portion of a first one of the plurality of images generated while the optical system is positioned at a first position with respect to the vicinity,
measure a second brightness of a portion of a second one of the plurality of images generated while the optical system is positioned at a second position with respect to the vicinity, the second position different from the first position, wherein the portion of the second one of the images generally corresponds to the portion of the first one of the images, and
calculate a distance to the vicinity, responsively to the first and second brightnesses.

117. Apparatus for use in a lumen, comprising:
a projecting device, comprising two non-overlapping light sources at a known distance from one another, the projecting device configured to project, from the respective light sources, two non-parallel beams at an angle with respect to one another, onto an imaging area within the lumen;
an optical system, configured to enable forward and omnidirectional lateral viewing and to generate an image of the imaging area; and
a control unit, configured to:
detect respective spots of light generated by the beams in the generated image, and
responsively to the known distance, an apparent distance between the spots, and the angle, calculate a distance to a vicinity of an object of interest of the lumen within the imaging area.

118. A method for use in a lumen, comprising:
illuminating a vicinity of an object of interest of a wall of the lumen;
generating a first omnidirectional image and a second omnidirectional image of the vicinity from a first position and a second position, respectively, the second position different from the first position;
measuring a first brightness of a portion of the first image, and a second brightness of a portion of the second image, the portion of the second image generally corresponding to the portion of the first image; and
calculating a distance to the vicinity, responsively to the first and second brightnesses.

119. A method for use in a lumen, comprising:
illuminating a vicinity of an object of interest of a wall of the lumen;
generating an omnidirectional image of the vicinity;
assessing a distortion of the image; and
calculating a distance to the vicinity responsively to the assessing.

120. A method for use in a lumen, comprising:
inserting into the lumen an optical system configured to enable forward and omnidirectional lateral viewing;
illuminating a vicinity of an object of interest of a wall of the lumen;
using the optical system, generating a first omnidirectional image of the vicinity while the optical system has a first focal length, and a second omnidirectional image of the vicinity while the optical system has a second focal length, different from the first focal length;
measuring a first magnification of a portion of the first image generated while the optical system has the first focal length, and a second magnification of a portion of the second image generated while the optical system has the second focal length, the portion of the second image generally corresponding to the portion of the first image;
comparing the first and second magnifications; and
calculating a distance to the vicinity, responsively to the comparison.

121. A method for use in a lumen, comprising:
inserting into the lumen an optical system configured to enable forward and omnidirectional lateral viewing;
illuminating a vicinity of an object of interest of a wall of the lumen;
using the optical system, generating a first omnidirectional image of a portion of the vicinity while the optical system has a first focal length, and a second omnidirectional image of the portion while the optical system has a second focal length;
comparing respective apparent sizes of the first and second images of the portion generated while the optical system has the first and second focal lengths, respectively; and
calculating a distance to the vicinity, responsively to the comparison.

122. A method for use in a lumen, comprising:
projecting a projected pattern onto an imaging area within the lumen;
generating an omnidirectional image of the imaging area;
detecting a pattern in the generated image;
analyzing the detected pattern;
responsively to the analysis, calculating a parameter selected from the group consisting of: a distance to a vicinity of an object of interest of the lumen within the imaging area, and a size of the object of interest.

123. A method for use in a lumen, comprising:
projecting a beam onto an imaging area within the lumen, the beam having a known size at its point of origin, and a known divergence;
generating an omnidirectional image of the imaging area;
detecting a spot of light generated by the beam in the generated image; and
responsively to an apparent size of the spot, the known beam size, and the known divergence, calculating a distance to a vicinity of an object of interest of the lumen within the imaging area.

124. A method for use in a lumen, comprising:
projecting, from two non-overlapping positions within the lumen at a known distance from one another, two respective non-parallel beams at an angle with respect to one another, onto an imaging area within the lumen;
generating an omnidirectional image of the imaging area;
detecting respective spots of light generated by the beams in the generated image; and
responsively to the known distance, an apparent distance between the spots, and the angle, calculating a distance to a vicinity of an object of interest of the lumen within the imaging area.
